(19) United States
(12) Patent Application Publication
(10) Pub. No.: US 2025/0000439 A1
(43) Pub. Date: Jan. 2, 2025

(54) METHOD FOR CONTROLLING COSMETIC APPARATUS, HEAD-MOUNTED DEVICE, AND COMPUTER-READABLE STORAGE MEDIUM

(71) Applicant: Flossom (GD) Beauty Technology Co., Ltd., Shunde District (CN)

(72) Inventors: Hairu JIANG, Shunde District (CN); Dongdong QIAN, Shunde District (CN); Xuefeng WANG, Shunde District (CN); Jialiang TAN, Shunde District (CN); Huibin WANG, Shunde District (CN); Mingyu CHEN, Shunde District (CN)

(21) Appl. No.: 18/734,073

(22) Filed: Jun. 5, 2024

Related U.S. Application Data
(63) Continuation of application No. PCT/CN2023/133672, filed on Nov. 23, 2023.

(30) Foreign Application Priority Data
Jun. 30, 2023 (CN) 202310797671.4

(51) Int. Cl. A61B 5/00; G16H 50/20

(52) U.S. Cl. CPC A61B 5/443; A61B 5/6803; A61B 5/6814; G16H 50/20

(57) ABSTRACT

A method for controlling a cosmetic apparatus, a head-mounted device, and a computer-readable storage medium. The method includes: sending a care mode request; obtaining a care mode that is recommended in response to the care mode request; identifying a history of the cosmetic apparatus using the recommended care mode; controlling, if the recommended care mode is determined as a historically used care mode, controlling the cosmetic apparatus to care for a user's face based on the recommended care mode; obtaining, if the recommended care mode is determined as a care mode that is not used in the history, status information on skin of the user's face; adjusting the recommended care mode based on the status information on the skin of the user's face; and controlling the cosmetic apparatus to care for the user's face based on the recommended care mode that has been adjusted.

### CROSS-REFERENCE TO RELATED APPLICATION

The present application is a continuation of PCT Application No. PCT/CN2023/133672, filed on Nov. 23, 2023, which claims the priority to Chinese patent Application No. 202310797671.4 entitled “METHOD FOR CONTROLLING COSMETIC APPARATUS, HEAD-MOUNTED DEVICE, AND COMPUTER-READABLE STORAGE MEDIUM” and filed with the China National Intellectual Property Administration on Jun. 30, 2023, the entireties of all of which are incorporated herein by reference.

### FIELD

The present disclosure relates to the technology field of cosmetic apparatuses, and more particularly, to a method for controlling a cosmetic apparatus, a head-mounted device, and a computer-readable storage medium.

### BACKGROUND

With the progress and development of society, people's pursuit of beauty is getting higher and higher. In view of people's love of beauty, a variety of beauty and skincare instruments appear accordingly and are widely approved by people. With the rapid popularization of cosmetic apparatuses, people have higher and higher requirements on cosmetic apparatuses. For a conventional cosmetic apparatus, a user usually selects a designated tap position through a button so as to perform a cosmetic operation. However, this is not intelligent enough. A small number of cosmetic apparatuses can detect a user's skin condition when in use, so as to recommend a suitable care plan or automatically perform an adaptive care mode based on the specific condition of the user's skin and thus to satisfy the user's beauty and care needs.

In the related technology, although a cosmetic apparatus can detect a skin condition of a user, but can only detect the current skin condition of the user at the time of the cosmetic apparatus is being used by the user, and can not consider the effect of the environment where the user is located on the skin of the user. Alternatively, even if the environment where the user is located is taken into account, the environmental parameter taken into account is only limited to data in a coarse range, which cannot be accurate to the user's activity trajectory. Therefore, there is often data deviation, and it is impossible to provide the user with a more suitable and more complete beauty plan.

### SUMMARY

The present disclosure aims to solve at least one of the technical problems existing in the prior art. Therefore, one purpose of the present disclosure is to propose a method for controlling a cosmetic apparatus, which considers the impact of information on an activity trajectory of the user and the environment where the user is located on the skin, and can provide the user with a more suitable and complete beauty plan that is more intelligent and can meet the user's beauty and care needs.

A second purpose of the present disclosure is to propose a head-mounted device.

A third purpose of the present disclosure is to propose a non-transitory computer-readable storage medium.

In order to solve the above problems, an embodiment in a first aspect of the present disclosure provides a method for controlling the cosmetic apparatus, including: sending a care mode request; obtaining a care mode that is recommended in response to the care mode request; identifying a history of the cosmetic apparatus using the recommended care mode; controlling, if the recommended care mode is determined as a historically used care mode, the cosmetic apparatus to care for a user's face based on the recommended care mode; obtaining, if the recommended care mode is identified as a care mode that is not used in the history, status information on skin of the user's face; adjusting the recommended care mode based on the status information on the skin of the user's face; and controlling the cosmetic apparatus to care for the user's face based on the recommended care mode that has been adjusted.

In the method for controlling the cosmetic apparatus according to the embodiment of the present disclosure, a head-mounted device is provided, and the head-mounted device can be worn by the user for a long time when the user is in life, at work or engages in an outside activity. The head-mounted device can not only fix the hair, but also have a decorative function. The head-mounted device can obtain the information on the activity trajectory of the user and the environment where the activity trajectory is located, and analyze the degree of influence of the activity trajectory and the environmental parameter on the skin, so as to determine a target care mode. After the head-mounted device is connected with the cosmetic apparatus (such as an electronic mask), the target care mode can be sent to the cosmetic apparatus. The cosmetic apparatus performs the target care mode for the user's care. The control method of this embodiment fully considers the influence of the information on the activity trajectory of the user and the environment where the activity trajectory is located on the skin. The reference data is comprehensive, and thus a more suitable and more perfect beauty plan can be provided to the user. The beauty plan is more intelligent and meets the user's needs for beauty and care.

According to some specific embodiments of the present disclosure, the obtaining the care mode that is recommended in response to the care mode request includes: obtaining a care mode that is recommended by a mobile terminal based on a habit of the user using the cosmetic apparatus; or obtaining a care mode that is generated by the mobile terminal based on a statistical care improvement report; or obtaining a care mode that is generated by the mobile terminal based on care information set by the user; or obtaining a care mode that is recommended by the mobile terminal based on recommendations of others for using the cosmetic apparatus for skin care.

According to some specific embodiments of the present disclosure, the method for controlling the cosmetic apparatus further includes: obtaining current seasonal information if the recommended care mode is determined as the care mode that is not used in the history; and adjusting a care parameter of the recommended care mode based on the seasonal information.

According to some specific embodiments of the present disclosure, in the method for controlling the cosmetic apparatus, the seasonal information includes weather information. The adjusting the care parameter of the recommended care mode based on the seasonal information includes: adjusting a moisturizing parameter and a whitening parameter in the recommended care mode based on the weather information.

According to some specific embodiments of the present disclosure, the method for controlling the cosmetic apparatus further includes: obtaining age and gender of the user if the recommended care mode is determined as the care mode that is not used in the history; and adjusting a control parameter of the recommended care mode based on the age and gender of the user.

According to some specific embodiments of the present disclosure, the method for controlling the cosmetic apparatus further includes: obtaining, if the recommended care mode is determined as the care mode that is not used in the history, an activity trajectory of the user and an environmental parameter around the activity trajectory; adjusting a control parameter of the recommended care mode based on the activity trajectory of the user and the environmental parameter around the activity trajectory.

According to some specific embodiments of the present disclosure, in the method for controlling the cosmetic apparatus, the obtaining the activity trajectory of the user includes: obtaining information on the activity trajectory of the user on the day that is collected by a mobile terminal.

According to some specific embodiments of the present disclosure, in the method for controlling the cosmetic apparatus, the information on the activity trajectory of the user for the day includes at least one of activity calories, exercise duration, activity duration, or total activity step number.

According to some specific embodiments of the present disclosure, the method for controlling the cosmetic apparatus further includes: sending the recommended care mode that has been adjusted and seasonal information, age information, gender information and environmental information corresponding to use time of the cosmetic apparatus to a mobile terminal or a cloud.

According to some specific embodiments of the present disclosure, in the method for controlling the cosmetic apparatus, the recommended care mode is a target care mode when the recommended care mode is determined as the historically used care mode, the recommended care mode that has been adjusted is a target care mode when the recommended care mode is determined as the care mode that is not used in the history. The method for controlling the cosmetic apparatus further includes: obtaining information on the user's face before using the cosmetic apparatus to care for the user based on the target care mode; obtaining information on the user's face after using the cosmetic apparatus to care for the user based on the target care mode; generating a care improvement report based on the information on the user's face before the care and the information on the user's face after the care; and sending the care improvement report to the mobile terminal.

According to some specific embodiments of the present disclosure, the cosmetic apparatus includes one of a handheld cosmetic apparatus, an electronic mask, or a beauty mask.

An embodiment in a second aspect of the present disclosure provides a head-mounted device. The head-mounted device includes: a communication module communicatively connected to a mobile terminal; at least one processor; and a memory. The memory is communicatively connected to the at least one processor. The memory stores a computer program that is executable by the at least one processor. The computer program when executed by the at least one processor, implements the method for controlling the cosmetic apparatus according to any of the above embodiments.

The head-mounted device according to the embodiment of the present disclosure fully considers the impact of the user's activity trajectory information and the environment where the activity trajectory is located on the skin, and the reference data is comprehensive. Therefore, a more suitable and more perfect beauty plan can be provided to the user, and the beauty plan is more intelligent and meets the user's needs for beauty and care.

Further, the head-mounted device is a smart hair band.

An embodiment in a third aspect of the present disclosure provides a non-transitory computer-readable storage medium stored with a computer program thereon. The computer program when executed, implements the method for controlling the cosmetic apparatus in any of the above embodiments. The method fully considers the impact of the information on the activity trajectory of the user and the environment where the activity trajectory is located on the skin. Therefore, a more suitable and more perfect beauty plan is provided to the user, and the beauty plan is more intelligent and meets the user's needs for beauty and care.

The additional aspects and advantages of this disclosure will be provided in the following description, which will become apparent from the following description, or will be learned through the practice of this application.

### BRIEF DESCRIPTION OF DRAWINGS

The above and/or additional aspects and advantages of the present disclosure will become apparent and easy to understand from the description of embodiments in conjunction with the accompanying drawings, in which:

FIG. 1 is a flowchart of a method for controlling a cosmetic apparatus according to an embodiment of the present disclosure;

FIG. 2 is a flowchart of a method for controlling a cosmetic apparatus according to another embodiment of the present disclosure;

FIG. 3 is a flowchart of a method for controlling a cosmetic apparatus according to still another embodiment of the present disclosure;

FIG. 4 is a flowchart of a method for controlling a cosmetic apparatus according to yet another embodiment of the present disclosure;

FIG. 5 is a block diagram of a head-mounted device, a cosmetic apparatus and a mobile terminal according to an embodiment of the present disclosure;

FIG. 6 is a block diagram of a head-mounted device, a cosmetic apparatus and a mobile terminal according to another embodiment of the present disclosure;

FIG. 7 is a block diagram of a head-mounted device according to an embodiment of the present disclosure.

### REFERENCE NUMERALS

Head-mounted device 10, Cosmetic apparatus 20, Mobile terminal 30,
Memory 102,
Communication module 1, Activity trajectory detection module 2, Environment detection module 3, Processor 4.

### DETAILED DESCRIPTION

Embodiments of the present disclosure are described in detail below, and examples of the embodiments are shown in the attached drawings. Same or similar labels in the whole description indicate the same or similar elements or elements having the same or similar functions. The embodiments described below with reference to the attached drawings are exemplary and are used only for the purpose of explaining the present application and are not to be construed as limitations to the present disclosure.

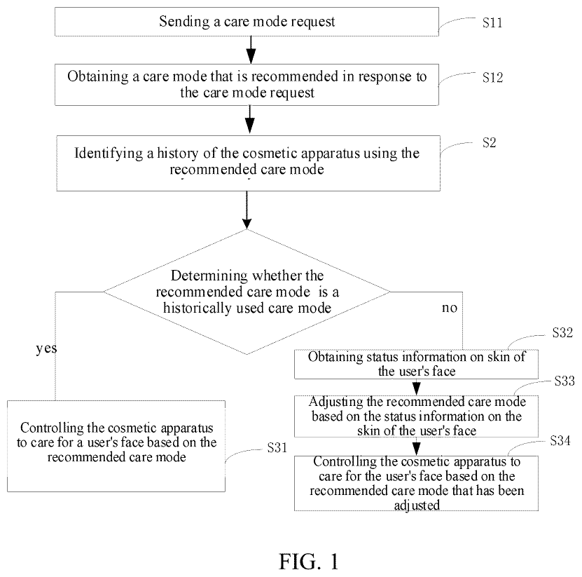

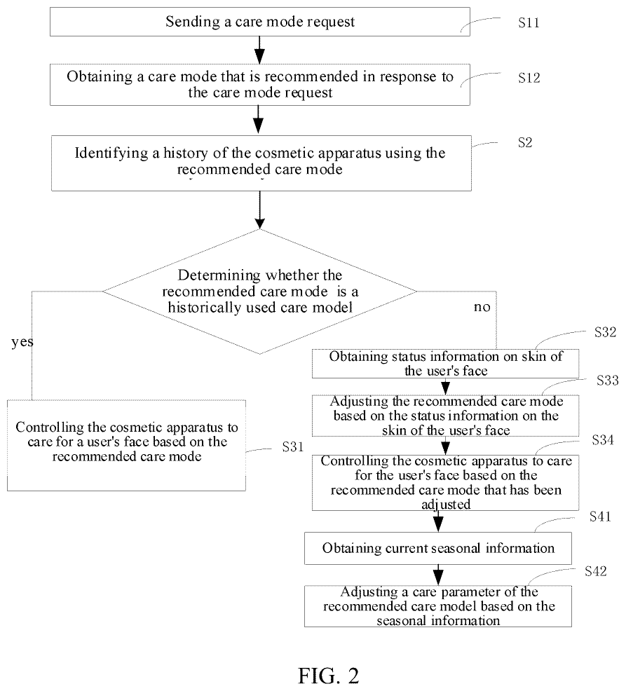

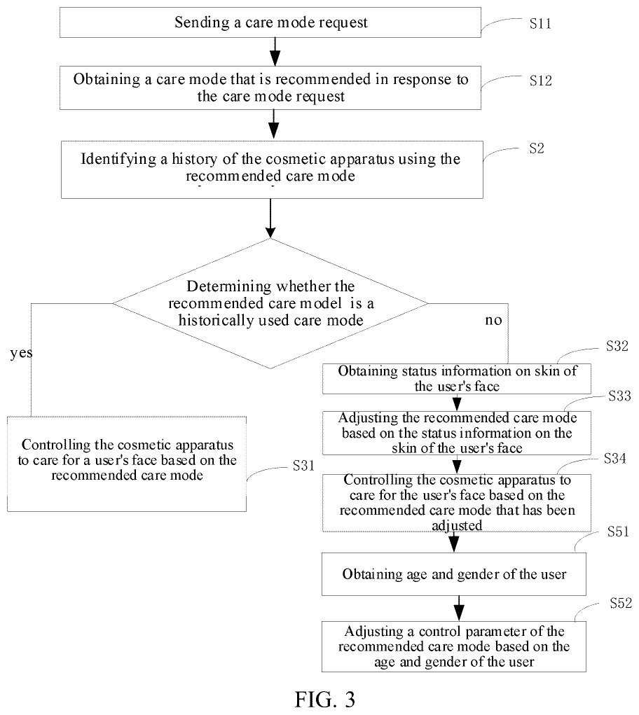

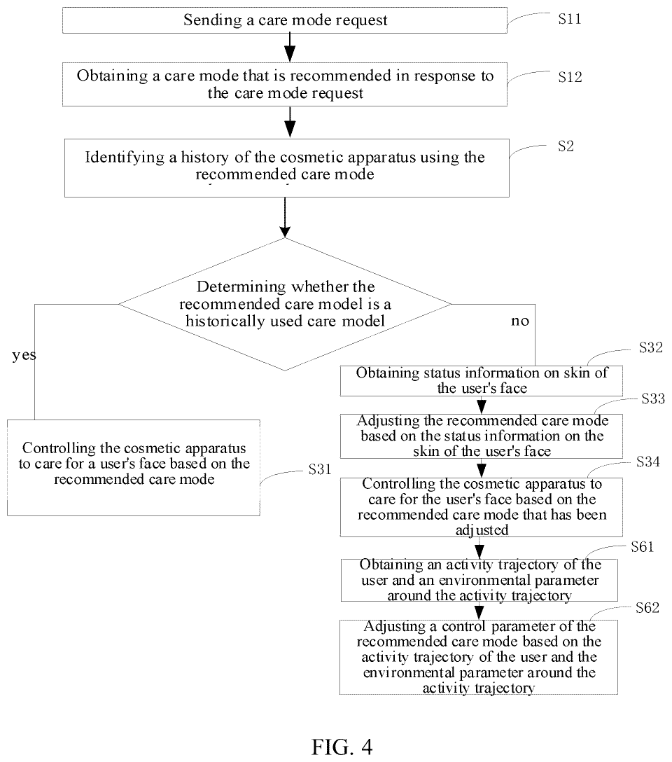

A method for controlling a cosmetic apparatus according to an embodiment of the present application is described below with reference to FIGS. 1 to 6.

FIG. 1 shows a flowchart of a method for controlling a cosmetic apparatus according to an embodiment of the present disclosure. The method for controlling the cosmetic apparatus includes at least steps S11-S42, as follows.

At S11, a care mode request is sent.

At S12, a care mode that is recommended in response to the care mode request is obtained.

At S2, a history of the cosmetic apparatus using the recommended care mode is identified.

At S31, if the recommended care mode is determined as a historically used care mode, the cosmetic apparatus is controlled to care for a user's face based on the recommended care mode.

At S32, if the recommended care mode is determined as a care mode that is not used in the history, status information on skin of the user's face is obtained.

At S33, the recommended care mode is adjusted based on the status information on the skin of the user's face.

At S34, the cosmetic apparatus is controlled to care for the user's face based on the recommended care mode that has been adjusted.

For example, the cosmetic apparatus sends a care mode request. An electronic device, such as a head-mounted device or a mobile terminal, obtains a care mode that is recommended in response to the care mode request. The electronic device records the care mode adopted by the cosmetic apparatus in its each operation, and identifies a history of the cosmetic apparatus using the recommended care mode. When the cosmetic apparatus sends a care mode request, another electronic device communicates with the cosmetic apparatus to obtain a corresponding recommended care mode and sends the recommended care mode to the user for the user to choose whether to adopt it.

For example, obtaining the care mode that is recommended in response to the care mode request may include, but is not limited to: obtaining a care mode that is recommended based on a habit of the user using the cosmetic apparatus; or obtaining a care mode that is generated based on a statistical care improvement report; or obtaining a care mode that is generated based on care information set by the user; or obtaining a care mode that is recommended based on recommendations of others for using the cosmetic apparatus for skin care.

When the recommended care mode is a historically used care mode, i.e., a previously recommended care mode, the use of the historically used care mode is more suitable for the user's daily care, and thus it is sufficient to recommend the user to keep using the historically used care mode. At this time, the electronic device sends a signal of the recommended care mode to the cosmetic apparatus and controls the cosmetic apparatus to care for the user's face. It is understood that at this time, the recommended care mode can be regarded as a target care mode.

When the recommended care mode is a care mode that is not used in the history, that is, the care mode has not been recommended before, it is possible that status information on the user's facial skin has changed significantly over a long period of time, and it is necessary to reassess the user's face. At this time, the status information on the user's facial skin can be obtained to detect the way of the skincare mode, such as sunscreen, moisturizing, whitening, and other care modes. The recommended care mode can be adjusted based on the status information on the user's facial skin, and a new care mode may be recommended to the user to satisfy the user's fresh care experience. It is understood that, the recommended care mode that has been adjusted can be regarded as a target care mode at this time.

The history of the cosmetic apparatus using the recommended care mode can be identified by the head-mounted device or the mobile terminal, and a history of a care mode used daily by the cosmetic apparatus is synchronously sent to the head-mounted device or the mobile terminal. The electronic device such as the head-mounted device or the mobile terminal determines whether the recommended care mode is a historically used care mode of the cosmetic apparatus. When it is determined that the recommended care mode is a care mode that is not used in the history, the electronic device such as the head-mounted device or the mobile terminal can send a command to the cosmetic apparatus to cause the cosmetic apparatus to obtain status information on skin of the user's face. After obtaining the status information on the skin of the user's face, the cosmetic apparatus sends again the status information on the skin of the user's face to the electronic device such as the head-mounted device or the mobile terminal. The electronic device such as the head-mounted device or the mobile terminal adjusts the recommended care mode based on the status information on the skin of the user' face, and controls the cosmetic apparatus to use the recommended care mode that has been adjusted to care for the user's face.

In this way, after each certain period of time, the care mode that is recommended in response to the care mode request may be a care mode that is not used in the history, and the status information on the skin of the user' face is reevaluated. The recommended care mode is adjusted, and the recommended care mode that has been adjusted is used to control the cosmetic apparatus to care for the user's face, so as to be adaptive to the user's care needs in different periods and under different environments.

Based on the care improvement report obtained statistically in the user's daily care process, a care mode that is more suitable to the user can be obtained. The care mode that is generated utilizing the powerful data processing function of the mobile terminal can include: a recommended cosmetic apparatus, a recommended tap position of the care currently performed by the cosmetic apparatus, such as an output power, a running frequency, a running time period, temperature and humidity and other information of the cosmetic apparatus. The mobile terminal can output a different power and a different running time period based on the user's habits of using the cosmetic apparatus, so as to achieve the best beauty and skincare effect on the user.

Further, the mobile terminal can also generate a care mode based on the statistical care improvement report, track the user's skincare process in real time via the generated care improvement report, and optimize the care mode, so as to make the recommended care mode more adaptive to the long-term trend of skincare, and gradually enhance and improve the user's skin health.

Further, the mobile terminal can also generate a care mode based on care information set by the user, that is, the user can personally select a care mode according to his own preference in the mobile terminal to meet the personalized needs of different users. The user can make a free choice. For example, the user can autonomously input the user's age, health status information, skin type, etc., and the mobile terminal generates a personalized care mode based on the care information set by the user. The mobile terminal can also combine the statistical data of the mobile terminal itself and the care information set by the user, and synthesize various care data to generate a care mode that is more suitable for the user.

Further, the mobile terminal can also recommend a care mode based on the recommendations of others for using the cosmetic apparatus for skin care. For example, a user using the head-mounted device and the mobile terminal can upload their care mode to the cloud, and other users can directly obtain the care mode in the cloud through their mobile terminals with the help of the networking module, thereby realizing the sharing of care mode.

In some specific embodiments of the present disclosure, the method for controlling the cosmetic apparatus further includes the follow steps of:
S41, obtaining current seasonal information; and
S42, adjusting a care parameter of the recommended care mode based on the seasonal information.

For example, an electronic device such as a head-mounted device or a mobile terminal can analyze the current season and adjust a care parameter so as to recommend a care mode (that is, the target care mode) more suitable for the current season to the user. The cosmetic apparatus operates based on the recommended care mode and automatically performs skincare for the user. Further, the seasonal information includes weather information. A moisturizing parameter and a whitening parameter in the recommended care mode are adjusted based on the weather information to obtain the target care mode. The target care mode can include a mode such as a cleaning mode, a moisturizing mode, a brightening mode, or a massaging mode. In different care modes, the temperature at an end of the cosmetic apparatus or care levels such as ionic strength level 1, level 2, or level 3 can be determined, in order to provide the user with a suitable beauty service. A different moisturizing parameter and a different whitening parameter are set in a different mode such as the cleaning mode, the moisturizing mode, the skincare or massaging mode. For example, a higher moisturizing parameter is set in winter having a dry environments to enhance additional water amount. A higher whitening parameter is set in summer having a higher UV environment, so as to further adjust the recommended care mode to make the care mode more complete.

In addition, in some embodiments, the method for controlling the cosmetic apparatus further includes steps S51 and S52, as follows:
S51, obtaining age and gender of the user; and
S52, adjusting a control parameter of the recommended care mode based on the age and gender of the user.

For example, an electronic device such as a head-mounted device or a mobile terminal can adjust a control parameter of the care mode with reference to the age and gender of the user, and recommend, for the user, a care mode that is more suitable for the age and gender of the user (that is, the target care mode). The cosmetic apparatus can run based on the target care mode, and automatically perform skin care for the user.

For users of different ages and genders, there are large differences in skin status. The cosmetic apparatus set different care modes for people of different ages and different genders, and the whitening parameter and the moisturizing parameter are different among different care modes.

In some specific embodiments of the present disclosure, the method for controlling the cosmetic apparatus further includes step S6, as follows:

S6, obtaining, if the recommended care mode is determined as the care mode that is not used in the history, an activity trajectory of the user and an environmental parameter around the activity trajectory; and adjusting a control parameter of the recommended care mode based on the activity trajectory of the user and the environmental parameter around the activity trajectory.

Specifically, the control parameter of the care mode can be adjusted based on the degree of influence of the activity trajectory of the user and the environmental information on the skin. For example, in an environment with a high illumination intensity, the skin is stimulated by UV irritation and sweating can easily cause skin problems, thus the skin should be cleaned timely. At this time, the user can be prompted to use mild cleaning products. When the user exercises in different environments, timely moisturizing should be performed, and the user can be prompted to use lotion or cream containing moisturizing ingredients to help the skin to maintain moisture. Balanced nutrition should be guaranteed for human body after exercise, and the human body needs to get adequate proteins, vitamins and other nutrients. Therefore, the user can be prompted to appropriately use vitamin-containing skincare products. The targeted care mode can be determined based on the time length in which the skin is exposed to environments such as a hot and dry environment, a ultraviolet irradiation environment, and a harmful substance environment, as well as based on the intensity and dose of UV rays and the concentration of harmful substances.

In other embodiments of the present disclosure, obtaining the activity trajectory of the user and the environmental parameter around the activity trajectory includes at least one of: collecting a UV illumination intensity, an irradiation duration, and a UV dose of UV rays in an environment where the activity trajectory of the user is located; collecting harmful substances and concentrations of these harmful substances in the air of the environment where the activity trajectory of the user is located; or collecting temperature and humidity of the air in the environment where the activity trajectory of the user is located.

A UV sensor can be provided in the head-mounted device for collecting UV illumination intensity or UV dose. A PM2.5 sensor can also be provided in the head-mounted device for collecting harmful substances and concentrations of these harmful substances in the air of the environment where the activity trajectory of the user is located, so as to calculate the quality of the air to which the user is exposed in that day. A temperature and humidity sensor can also be provided in the head-mounted device for collecting the temperature and humidity of the air in the environment where the activity trajectory of the user is located.

Specifically, UV illumination intensity (i.e., UV irradiance) is a physical quantity used to describe the intensity of UV radiation, in watts per square meter (W/m²). The UV irradiance is usually used to measure the intensity of a UV lamp or solar radiation. UV dose is used to describe a total amount of UV radiation to which a person is exposed, and it is usually used to study the effects of UV radiation on the skin. The unit of the UV dose is joules per square meter (J/m²) or megajoules per square meter (MJ/m²). The UV dose is closely related to its effect on human skin. In general, a higher UV dose leads to a more obvious effect on the skin.

Further, the level of PM2.5 concentration of harmful substances is closely related to its effect on human skin. A higher concentration of PM2.5 in the air means more PM2.5 particulates in the air and is more harmful to human health.

Further, air temperature and humidity can affect the moisture content of the skin and the skin barrier function, thereby can affect the health and appearance of the skin. For example, in a low-humidity environment, the low moisture content in the air may cause the skin to lose moisture and become dry, rough, and itchy. Furthermore, the barrier function of the skin in a low-humidity environment may be affected, resulting in the skin being more susceptible to be irritated and infected. As a result, collecting the temperature and humidity of the air can predict the degree of effect of the air temperature and humidity on the human skin.

Further, collecting the activity trajectory of the user wearing the head-mounted device includes obtaining information on the activity trajectory of the user on the day that is collected by the mobile terminal. The information on the activity trajectory of the user on the day includes at least one of activity calories, exercise duration, activity duration, or total activity step number. For example, an IMU can be provided in the head-mounted device for measuring acceleration and angular velocity. A pressure sensor can also be provided in the head-mounted device for measuring atmospheric pressure. By collecting a variety of types of data about the activity trajectory, more accurate information on the activity trajectory can be obtained. The parameter of the care mode can be adjusted based on the activity trajectory of the user and environmental information to obtain the target care mode.

In some embodiments of the present disclosure, the method for controlling the cosmetic apparatus further includes sending the recommended care mode that has been adjusted and seasonal information, age information, gender information, and environmental information corresponding to use time of the cosmetic apparatus to a mobile terminal or a cloud.

For example, the head-mounted device (such as a smart hair band) is utilized to obtain the seasonal information, the age information, the gender information, and the environmental information, and send them to the mobile terminal or the cloud. The mobile terminal recommends a care mode for the user through algorithms of an internal APP, provides a suggestion for recommending this care mode to the user, or directly controls the cosmetic apparatus to perform care based on the recommended care mode.

After the mobile terminal receives the seasonal information, the age information, the gender information, and the environmental information, can correct the recommended care mode based on the received seasonal information, age information, gender information, and environmental information, and generates an adjusted care mode. The adjusted care mode is more suitable to the user. Moreover, the seasonal information, the age information, the gender information, and the environmental information of the user and the target care mode can also be displayed on the mobile terminal, and the user can review in real time the seasonal information, the age information, the gender information, and the environmental information and the care status of the target care mode.

In some embodiments, the cosmetic apparatus includes one of a handheld cosmetic apparatus, an electronic mask, or a beauty mask. Specifically, based on the characteristics of each type of cosmetic apparatus, the corresponding output tap position and/or working mode under the target care mode can be set, and the output power and the working period under the corresponding mode can be sent to the corresponding cosmetic apparatus to meet the user's skin care requirements and enhance the user experience. The cosmetic apparatus can be used in conjunction with other electronic devices, which can bring more personalized care options for the user.

In some embodiments of the present disclosure, the method for controlling the cosmetic apparatus may further include: obtaining information on the user's face before using the cosmetic apparatus to care for the user based on the target care mode; and obtaining information on the user's face after using the cosmetic apparatus to care for the user based on the target care mode; and generating a care improvement report based on the information on the user's face before the care and the information on the user's face after the care; and sending the care improvement report to the mobile terminal. In the case where the recommended care mode is a historically used care mode, the recommended care mode is the target care mode; and in the case where the recommended care mode is a care mode that is not used in the history, the recommended care mode that has been adjusted is the target care mode.

That is, the status information of the user's facial skin care is obtained, the status information of the skin after the care is compared with the status information of the skin before the care, and the care improvement report is generated. The care improvement report can more intuitively shows the changes in the skin after the care. The report is provided to the user, so that the user can more intuitively know the effect of this care and improve the user's use experience. In addition, the skin care improvement report can also be sent to the cloud for reference by other users or for manufacturer improvement.

Further, in the embodiment, when the user uses the cosmetic apparatus (such as the electronic mask), user input information and the care improvement report can also be obtained, and a self-selected care mode of the user can be determined based on the user input information. The self-selected care mode of the user can be adjusted based on the care improvement report, information on the user's activity trajectory, information on the environment surrounding the activity trajectory, and the status information on the skin of the face, so as to determine the final target care mode and then to send the target care mode to the electronic mask. The electronic mask performs this target care mode to accurately control parameters. In this way, when the user's self-selected mode deviates from the actual situation, the care strategy can be adjusted timely, accurately and in a targeted way so as to achieve more effective care and improve the user's satisfaction and use experience.

A head-mounted device 10 according to an embodiment of the present disclosure will be described below.

The head-mounted device 10 according to the embodiment of the present disclosure includes a communication module 1, at least one processor 4, and a memory 102 that is communicatively connected to the at least one processor 4.

The communication module 1 is communicatively connected to the mobile terminal 30. A computer program executable by the at least one processor 4 is stored in the memory 102. The computer program when executed by the at least one processor 4, can implement the method for controlling the cosmetic apparatus according to any of the above embodiments.

Further, the head-mounted device 10 is a smart hair band. The user can wear the smart hair band for a long time when the user is at work or engages in an outside activity. The smart hair band can not only fix the hair, but also have a decorative function. The mobile terminal 30 can be an electronic product such as a mobile phone, a smartwatch, etc., A corresponding APP can be downloaded in the mobile terminal 30. The user can interconnect with the head-mounted device 10 and the cosmetic apparatus 20 through the APP. The mobile terminal 30 has the function of processing data. The mobile terminal 30 obtains a target care mode through data operation, and then sends the recommended care mode to the head-mounted device 10 through the corresponding APP, so as to enable a target cosmetic apparatus to care for the user's face based on the recommended care mode. The head-mounted device 10 is communicatively connected to the cosmetic apparatus 20. The cosmetic apparatus 20 may include a handheld cosmetic apparatus, an electronic mask, or a beauty mask, etc. There is no specific limitation on the connection between the head-mounted device 10 and the cosmetic apparatus 20. For example, when the cosmetic apparatus 20 is a beauty mask or an electronic mask, the head-mounted device 10 is provided with a connection portion, and the head-mounted device 10 can be electrically connected to the beauty mask or the electronic mask via the connection portion when the user is using the beauty mask or the electronic mask. As another example, when the cosmetic apparatus 20 is a handheld cosmetic apparatus, the head-mounted device 10 can be communicatively connected to the handheld cosmetic apparatus via Bluetooth when the user is using the handheld cosmetic apparatus.

The head-mounted device 10 can acquire the care mode performed by the cosmetic apparatus 20 in real time, so as to timely adjust the care mode based on the seasonal information, age information, gender information and environmental information of the user. When the seasonal information, the age information, the gender information and the environmental information change, the adjusted target care mode is recommended, or the current running care mode of the cosmetic apparatus 20 is refreshed in real time. The processor 4 provided in the head-mounted device 10 assists the mobile terminal 30 in making an adjustment to a care mode when the mobile terminal 30 recommends a care mode, so that a target care mode is jointly determined by the mobile terminal 30 and the head-mounted device 10, and the target care mode does not rely solely on the mobile terminal 30 or the head-mounted 10. Through the data operation performed jointly by the head-mounted 10 and the mobile terminal 30, the data processing function is powerful, which makes full use of the characteristics of the mobile terminal 30 and the head-mounted 10, and optimizes the care mode several times to generate a different care mode that more adaptive to the user's long-time care needs.

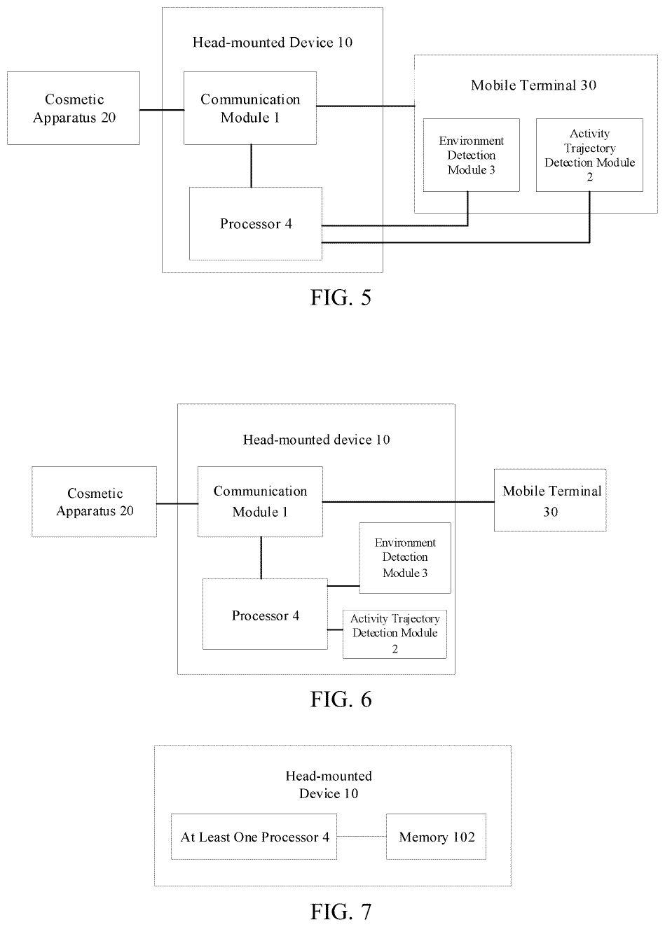

According to some embodiments of the present disclosure, as shown in FIG. 6, the head-mounted device 10 may further include an activity trajectory detection module 2 and an environment detection module 3. Alternatively, as shown in FIG. 5, the activity trajectory detection module 2 and the environment detection module 3 are disposed in the mobile terminal 30. The activity trajectory detection module 2 is configured to obtain an activity trajectory of the user. The environment detection module 3 is configured to obtain an environmental parameter around the activity trajectory of the user.

The non-transitory computer-readable storage medium provided according to the embodiment of the present disclosure stores a computer program thereon. The computer program when executed by the processor 4, may implement the method for controlling the cosmetic apparatus according to any of the above embodiments. By adopting the method, the impact of the user's activity trajectory information and the environment where the activity trajectory is located on the skin is fully considered. Therefore, a more suitable and more perfect beauty plan can be provided to the user, and the method is more intelligent and satisfy the user's needs for beauty and care.

Other constructors and operations of the cosmetic apparatus 20 according to the embodiment of the present disclosure are known to those of ordinary skill in the art and are not described in detail herein.

In the illustration of the description, an illustration with reference to the terms “one embodiment”, “some embodiments”, “illustrative embodiments”, “an example”, “a particular example” or “some examples” and so on mean that a particular feature, structure, material, or characteristic described in connection with the embodiment(s) or example(s) is included in at least one embodiment or example of the present disclosure. In this description, the exemplary expressions of the above terms do not necessarily specify the same embodiments or examples.

Although embodiments of the present disclosure have been shown and described, it will be understood by those skilled in the art that various changes, modifications, alternations and variations may be made to these embodiments without departing from the principles and spirit of the present disclosure, the scope of which is defined by the claims and their equivalents.

What is claimed is:

1. A method for controlling a cosmetic apparatus, comprising:
sending a care mode request;
obtaining a care mode that is recommended in response to the care mode request;
identifying a history of the cosmetic apparatus using the recommended care mode;
controlling, if the recommended care mode is determined as a historically used care mode, the cosmetic apparatus to care for a user's face based on the recommended care mode;
obtaining, if the recommended care mode is determined as a care mode that is not used in the history, status information on skin of the user's face;
adjusting the recommended care mode based on the status information on the skin of the user's face; and
controlling the cosmetic apparatus to care for the user's face based on the recommended care mode that has been adjusted.

2. The method for controlling the cosmetic apparatus according to claim 1, wherein said obtaining the care mode that is recommended in response to the care mode request comprises:
obtaining a care mode that is recommended based on a habit of the user using the cosmetic apparatus; or
obtaining a care mode that is generated based on a statistical care improvement report; or
obtaining a care mode that is generated based on care information set by the user; or
obtaining a care mode that is recommended based on recommendations of others for using the cosmetic apparatus for skin care.

3. The method for controlling the cosmetic apparatus according to claim 1, further comprising:
obtaining, if the recommended care mode is determined as the care mode that is not used in the history, current seasonal information; and
adjusting a care parameter of the recommended care mode based on the seasonal information.

4. The method for controlling the cosmetic apparatus according to claim 3, wherein the seasonal information comprises weather information, and said adjusting the care parameter of the recommended care mode based on the seasonal information comprises:
adjusting a moisturizing parameter and a whitening parameter in the recommended care mode based on the weather information.

5. The method for controlling the cosmetic apparatus according to claim 1, further comprising:
obtaining, if the recommended care mode is determined as the care mode that is not used in the history, age and gender of the user; and
adjusting a control parameter of the recommended care mode based on the age and gender of the user.

6. The method for controlling the cosmetic apparatus according to claim 1, further comprising:
obtaining, if the recommended care mode is determined as the care mode that is not used in the history, an activity trajectory of the user and an environmental parameter around the activity trajectory;
adjusting a control parameter of the recommended care mode based on the activity trajectory of the user and the environmental parameter around the activity trajectory.

7. The method for controlling the cosmetic apparatus according to claim 6, wherein said obtaining the activity trajectory of the user comprises:
obtaining information on the activity trajectory of the user on the day that is collected by a mobile terminal.

8. The method for controlling the cosmetic apparatus according to claim 7, wherein the information on the activity trajectory of the user on the day comprises at least one of activity calories, exercise duration, activity duration, or total activity step number.

9. The method for controlling the cosmetic apparatus according to claim 1, further comprising:
sending the recommended care mode that has been adjusted and seasonal information, age information, gender information and environmental information corresponding to use time of the cosmetic apparatus to a mobile terminal or a cloud.

10. The method for controlling the cosmetic apparatus according to claim 1, wherein,
the recommended care mode is a target care mode when the recommended care mode is determined as the historically used care mode,
the recommended care mode that has been adjusted is a target care mode when the recommended care mode is determined as the care mode that is not used in the history, and
the method for controlling the cosmetic apparatus further comprises:
obtaining information on the user's face before using the cosmetic apparatus to care for the user based on the target care mode;
obtaining information on the user's face after using the cosmetic apparatus to care for the user based on the target care mode;
generating a care improvement report based on the information on the user's face before the care and the information on the user's face after the care; and
sending the care improvement report to the mobile terminal.

11. The method for controlling the cosmetic apparatus according to claim 1, wherein the cosmetic apparatus comprises one of a handheld cosmetic apparatus, an electronic mask, or a beauty mask.

12. A head-mounted device, comprising:
a communication module communicatively connected to a mobile terminal;
at least one processor; and
a memory, the memory communicatively connected to the at least one processor, wherein the memory stores a computer program that is executable by the at least one processor, and the computer program when executed by the at least one processor, implements the method for controlling the cosmetic apparatus according to claim 1.

13. The head-mounted device according to claim 12, wherein the computer program when executed by the at least one processor implements:
obtaining a care mode that is recommended based on a habit of the user using the cosmetic apparatus; or
obtaining a care mode that is generated based on a statistical care improvement report; or
obtaining a care mode that is generated based on care information set by the user; or
obtaining a care mode that is recommended based on recommendations of others for using the cosmetic apparatus for skin care.

14. The head-mounted device according to claim 12, wherein the computer program when executed by the at least one processor implements:
obtaining, if the recommended care mode is determined as the care mode that is not used in the history, current seasonal information; and
adjusting a care parameter of the recommended care mode based on the seasonal information.

15. The head-mounted device according to claim 13, wherein the seasonal information comprises weather information, and the computer program when executed by the at least one processor implements:
adjusting a moisturizing parameter and a whitening parameter in the recommended care mode based on the weather information.

16. The head-mounted device according to claim 12, wherein the computer program when executed by the at least one processor implements:
obtaining, if the recommended care mode is determined as the care mode that is not used in the history, age and gender of the user; and
adjusting a control parameter of the recommended care mode based on the age and gender of the user.

17. The head-mounted device according to claim 12, wherein the computer program when executed by the at least one processor implements:
obtaining, if the recommended care mode is determined as the care mode that is not used in the history, an activity trajectory of the user and an environmental parameter around the activity trajectory;
adjusting a control parameter of the recommended care mode based on the activity trajectory of the user and the environmental parameter around the activity trajectory.

18. The head-mounted device according to claim 17, wherein the computer program when executed by the at least one processor, implements:
obtaining information on the activity trajectory of the user on the day that is collected by a mobile terminal.

19. The head-mounted device according to claim 12, wherein the head-mounted device is a smart hair band.

20. A non-transitory computer-readable storage medium stored with a computer program thereon, wherein the computer program when executed, implements the method for controlling the cosmetic apparatus according to claim 1.
